(19) United States
(12) Patent Application Publication
(10) Pub. No.: US 2009/0149698 A1
(43) Pub. Date: Jun. 11, 2009

(54) Weighted lap pad with sensory attachments

(76) Inventor: Cynthia L. Tastard, West Scramento, CA (US)

Correspondence Address: Cynthia L. Tastard, P.O. Box 2284, West Sacramento, CA, 95691, US

(21) Appl. No.: 12/316,093

(22) Filed: Dec. 8, 2008

Related U.S. Application Data
(60) Provisional application No. 61/005,964, filed on Dec. 8, 2007.

(51) Int. Cl. A61M 21/00; A63G 31/00

(52) U.S. Cl. 600/27; 472/133

(57) ABSTRACT

This kidney shaped, weighted lap pad is designed to have interchangeable items that stimulate sensory input (fidgets) attached to the top of it. This invention is designed to provide helpful sensory input to individuals with disabilities. Providing this input in a controlled manner inhibits the user from seeking sensory input in inappropriate ways; Thereby calming users, allowing them to be focused on other things, like a teacher's instruction. It comes in several sizes for people ages three years and up, and can weigh two to eight pounds. The lap pad has anchors sewn on the surface, to which fidgets are attached. Different removable attachment devices, such as key-ring loops, clips or carabineers, can be utilized. This design allows for fidgets manufactured for the lap pad to be interchangeable. The lap-pad also has a washable/removable outer cover, and straps at each end for carrying it.

### CROSS-REFERENCED TO RELATED APPLICATIONS

The benefit of provisional patent application Ser. No. 61/005,964 filed Dec. 8, 2007 is claimed.

### STATEMENT REGARDING FEDERALLY SPONSORED RESEARCH

Not Applicable

### NAMES OF THE PARTIES TO A JOINT RESEARCH AGREEMENT

Not Applicable

### INCORPORATION BY REFERENCE OF MATERIAL SUBMITTED ON A COMPACT DISC

Not Applicable

### BACKGROUND OF THE INVENTION

The field of the invention relates to weighted apparel which addresses the sensory needs of people with disabilities such as Attention Deficit Hyperactivity Disorder (ADHD), autism spectrum disorders, sensory integration disorders and other disabilities. Individuals with these conditions often have sensory integration problems which drive them to seek extra sensory stimulation by means of touching items and moving their bodies, activities which are often disruptive to learning, and contrary to social norms. This invention attempts to address two types of sensory stimulation needs: body position (proprioception) and touch (tactile). It was developed primarily as a non-invasive classroom accommodation aimed at inhibiting students with disabilities from seeking sensory stimulation in an inappropriate manner; thereby enabling disabled students to focus on learning. This invention can also be beneficial in the home and community, and to adults with similar disabilities.

Using weight to apply deep pressure to the body is known to satisfy a sensory craving in many persons with autism, ADHD, and other disorders which affect sensory processing, with the end result of calming and focusing the individual. This calming effect can be an important goal in educating children with attention deficit hyperactivity disorder (“ADHD”) and others with attention difficulties such as autism spectrum disorders. See VandenBerg, N. L.; Vol. 55, No. 6, AJOT 621-628, The Use of Weighted Vest to Increase On-Task Behavior in Children with Attention Difficulties and Fertel-Daly, D., Bedell, G., & Hinojosa, J. (2001). Vol. 55, No. 6, AJOT, 629-640, Effects of a Weighted Vest on Attention to Task and Self-Stimulatory Behaviors in Preschoolers With Pervasive Developmental Disorders. Further documentation includes books by Temple Grandin, PhD., research by Dr. A. Jean Ayres, and articles in occupational/physical therapy publications. The senses which especially benefit from this type of weighted stimulation are those of body position (proprioception), touch (tactile), and balance (vestibular). When stimuli received from the environment cannot be adequately controlled or integrated by the nervous system, there are frequently resulting behaviors which interfere with attention and learning. Therefore, in any setting where attention to task is required (particularly in an educational setting), a means of satisfying this need is very desirable.

Known products that attempt to meet this particular sensory need include: weighted vests, weighted stuffed animals, weighted blankets, weighted lap pads, and weighted therapy apparel. However, many of these items are not conducive to being used in a classroom or out in society at large (e.g. church, movie theatre, car rides), as they are too cumbersome or conspicuous. In addition, many weighed vests, wrist/ankle weights, and weighted lap pads are often bulky, and/or rectangular shaped, making them difficult to conform to the body. In an effort to be utilitarian, they are often plastic-covered, with straps and Velcro, making them aesthetically unappealing. In an educational environment, wearing these items sets the students apart from their peers and subsequently, getting them to utilize the product becomes problematic, (especially among children of average intelligence). In addition, the Velcro, belts and straps used for fastening these products to the body often become noise-makers and toys. In addition, weights of other products are often removable from a Velcro closured pouch, which is easily accessed by the child. This is problematic, as the Velcro turns into a noisy distraction, and once the pouch is open, weights are easily lost rendering the device useless. In addition, the use of a belt to secure a lap weight is contrary to the desired result of having the child be able to self-regulate his body. If the child stands up, the lap-weight is tied to him and moves with him, giving him little indication to sit down. It would be more useful perhaps, to have the weight lie on the lap, without the use of a belt. Thereby if the child stood up, he would feel the weight begin to slip off, and thereby catch it and sit back down.

While the foregoing expedients have been used in an attempt to address a disabled person's sensory cravings, they primarily address the need for deep pressure. Most aforementioned inventions stop short of addressing what is often one of the most problematic sensory hungers of individuals with ADHD, autism spectrum disorders, and sensory integration disorders: that of touch and self stimulation. Individuals with these disabilities often have a sensory need to touch things with their hands, or self-stimulate. This behavior can be problematic and even harmful at times. Children with ADHD commonly turn anything they have within their reach into a sensory object: pens, paper-clips, toys, etc.: Taking these items apart, breaking them, poking themselves or others. This behavior can be problematic for obvious reasons. In a similar manner, individuals with autism may self-stimulate by flapping their hands, snapping their fingers, using common items inappropriately, thereby calling attention to themselves and becoming a distraction to others. While the lap weight, U.S. Pat. No. 6,383,130 to Wade and Queen, may have a material cover that is tactilely pleasing, the item as a whole does not maintain the interest of the user.

One method of addressing the tactile sensory hunger of these individuals that is commonly utilized by teachers, occupational therapists and school psychologists, is to intentionally give the individual something simple to manipulate that is unique to their liking, such as a stress ball or small fidget; thereby, soothing their tactile sensory hunger, and allowing them to focus their attention on other tasks. However, due to the associated problems these individuals often have with impulsivity, organization, and/or motor control, these items often end up getting lost, rolling across the floor, or becoming projectiles, thereby causing a greater commotion. What would be helpful in this situation would be a means to attach a sensory item to a fixed or semi-fixed base, making it improbable to be lost, thrown or dropped. The apparatus should allow for the sensory items to be easily interchangeable, to provide the user with novelty, and/or to provide a new user with a sensory attachment of their liking.

Given these considerations, a need exists for a device which will satisfy both a child's and adult's craving for deep pressure while also satisfying their desire for tactile sensory stimulation. This device should be easily utilized in a classroom and home environment and be inconspicuous in nature. In addition, the device should be able to be conveniently transported and used in multiple settings (school, home, therapy sessions, riding in car, etc.). The device should be appealing and engaging for the user, and provide frequent novelty. To provide novelty and versatility, the device should have a system of interchangeable sensory attachments. The device should come in different sizes and weights to address the needs of people of all ages, and the shape of it should conform to the wearer, so as to not be bulky or obtrusive. The device body should have the weighted substance permanently enclosed, and not accessible, such that the closure devices (Velcro, zippers, buttons) do not have to be utilized, and the weight material does not get lost. Preferable, the cover of the device should be removable and washable, appealing to the eye, and have a pleasant texture to retain the interest of the user.

### BRIEF SUMMARY OF THE INVENTION

The present invention is a weighted lap pad, designed to be placed across the lap of a seated individual whose dysfunctional sensory system requires additional stimulation. The present invention is kidney-shaped, to conform to an individual's torso, while covering the lap. The lap-pad has channeling sewn into it. A weighting substance (such as sand, plastic or metal pellets, or natural materials) is evenly distributed throughout the article by being sewn into the channeling. Preferrably this is done in a sunburst pattern, divergent from the center. This design allows the lap-pad to yield and fold to the wearer's body better. By design, the invention is wide enough to easily curve over the outer side of each leg, thereby fitting more snuggly than prior designs. This invention is very low-profile, and can easily be utilized under a desk, thereby allowing the user to be inconspicuous. For carrying purposes, the invention has a handle sewn onto at least one end.

One of the most important objectives of this invention is to provide the user with a continually engaging and interesting method to have their tactile sensory needs met. This is done through providing the user with sensory-tools which are secured to the weighted lap pad, preventing the user from accidentally dropping, throwing, or losing the item. Versatility is key, as people with sensory difficulties often need novelty to stay interested in things. This invention provides great flexibility by utilizing an attachment anchor (such as a ring, clip, snap or button) secured to the top surface of the lap pad, to which sensory tools (fidgets: items that stimulate visual and tactile sensory input) can be attached and detached. Sensory-tools can then be secured to the anchoring device with an attachment device that opens and closes (such as a key ring loop, clip, snap, or button).

An assortment of sensory tools may be included with the weighted lap pad. The sensory items may come in a variety of shapes, sizes and textures, (e.g. bendable toys, squishy balls, soft pieces of material, stretchy toys, knobby hard plastic items, etc.) If needed, a sensory-tool may have a strap sewn to it, to which an attachment device that opens and closes is secured, so it can then be attached to the lap pad anchor. Some sensory-tools will not need this modification, and will be easily attached with an attachment device that simply opens and closes.

In regard to materials for construction, the lap pad is made of a strong, durable, no-stretch material (i.e. Denier nylon, denim, etc.). This is to prevent tearing or punctures. It may be double-layered. The strong fabric provides a durable base to which the handle/s and sensory attachment anchor/s are sewn. The invention also has a removable and washable cover that is preferably form fitting. The cover can be a variety of fabric types (such as fleece, cotton, nylon, etc.) and can be solid or pattered. The form-fitting cover opens vertically or horizontally on the bottom side, preferably in the center, like a pillow-sham, such that the weighted lap pad slides in. While no zippers, buttons or closures may be used, it is preferable to have said opening overlap in the back to ensure the retention of the device. On at least one end of the cover, there is a small opening in the seaming where the handle of the weighted lap pad may be pulled through to become accessible. The handle is used for ease of transportation. On the top side of the cover, there may be small reinforced openings for the anchors on the lap pad to be pulled through. Once the anchors are accessible, the sensory-tools may be attached. The cover may also have hand-sized pockets on the surface, into which the user can slip his hands.

### BRIEF DESCRIPTION OF THE SEVERAL VIEWS OF THE DRAWINGS

FIG. 1 shows a top plane view of the weighted lap pad (without cover) and without sensory tools attached. (The posterior side is an exact replica of the top plane view, except there are not anchors or anchor attachments.)

FIG. 2 shows the weighted lap pad with cover and sensory tools attached.

FIG. 3 shows the anterior side of the invention with the cover on, and the overlapped opening.

FIG. 4 shows the invention, used as designed, in the lap of a child. The child is manipulating the sensory tools.

### DETAILED DESCRIPTION OF THE INVENTION

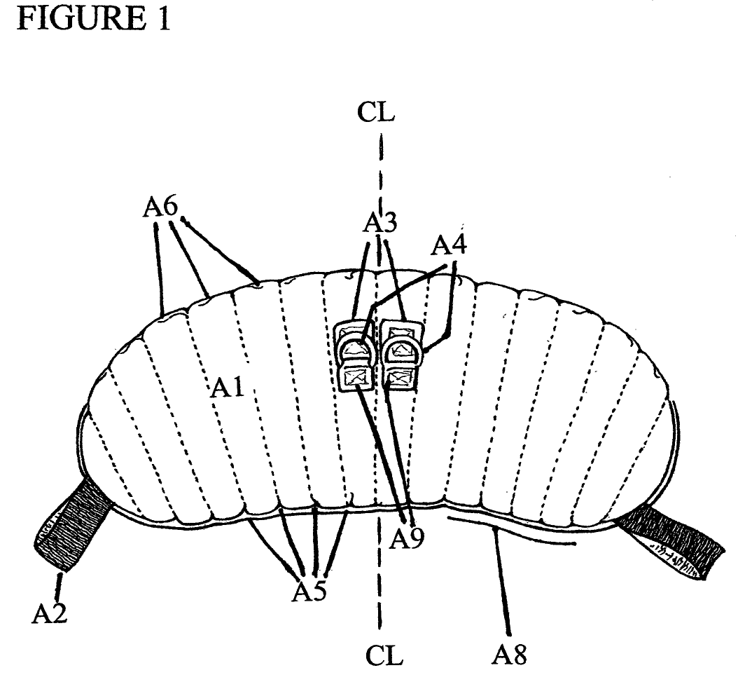

Referring to FIGS. 1, 2, 3, and 4, an embodiment of the invention is shown.

FIG. 1 illustrates the embodiment of the invention without its cover. This is a medium sized weighted lap pad with anchors for sensory tools. The invention is kidney shaped to conform to the body. The shape is symmetrical about the centerline CL. The material used for construction are two panels of no-stretch fabric (such as Cordura nylon or denim) A1, which resists tears and punctures. If desired for durability, a second layering of the same material or another material may be utilized. Seams A5 sewn through all layers, create 16 separate channels A6 which shorten towards the ends, into which a weighted substance is evenly distributed and sewn closed on the torso side A8. More or less channels may be used, depending upon the size of the device. The weighted substance may include, manmade or natural filler, preferably particulated with a smooth surface. The filled weighted lap pad is 1.0 inch thick +0.50/−0.50 inches. Weight will vary depending upon the fill substance. When standard play sand is used as a filler, this item weighs 4.0 lbs. +/−0.25 lbs. Two anchors A4 are centered symmetrically on the anterior side. The anchors may be 1.0 inch D-rings A4, retained with a 2.50 inch long piece of 1.00 inch wide web-belting A3 which may be sewn in an X-box pattern A9 on both sides of the D-ring. For carrying purposes, the invention has two webbing loops, which act as handles, sewn onto each end A2. The handles may be made of 8 inches of 1.00 inch wide belt-webbing, and are folded over to create an opening. The channels A6 allow the lap pad to articulate to the wearer's body. While a particular size is illustrated here, it should be noted again that the size and weight of this device may vary, according to the size and need of the user. Width (left to right measurement when the device is worn) of the device may be as wide as 36.0 inches, or as narrow as 15.0. Depth (knee to torso measurement) may be as deep as 12.0 inches or as thin as 5.0 inches. Weights may vary between 2.0 lbs and 10 lbs.

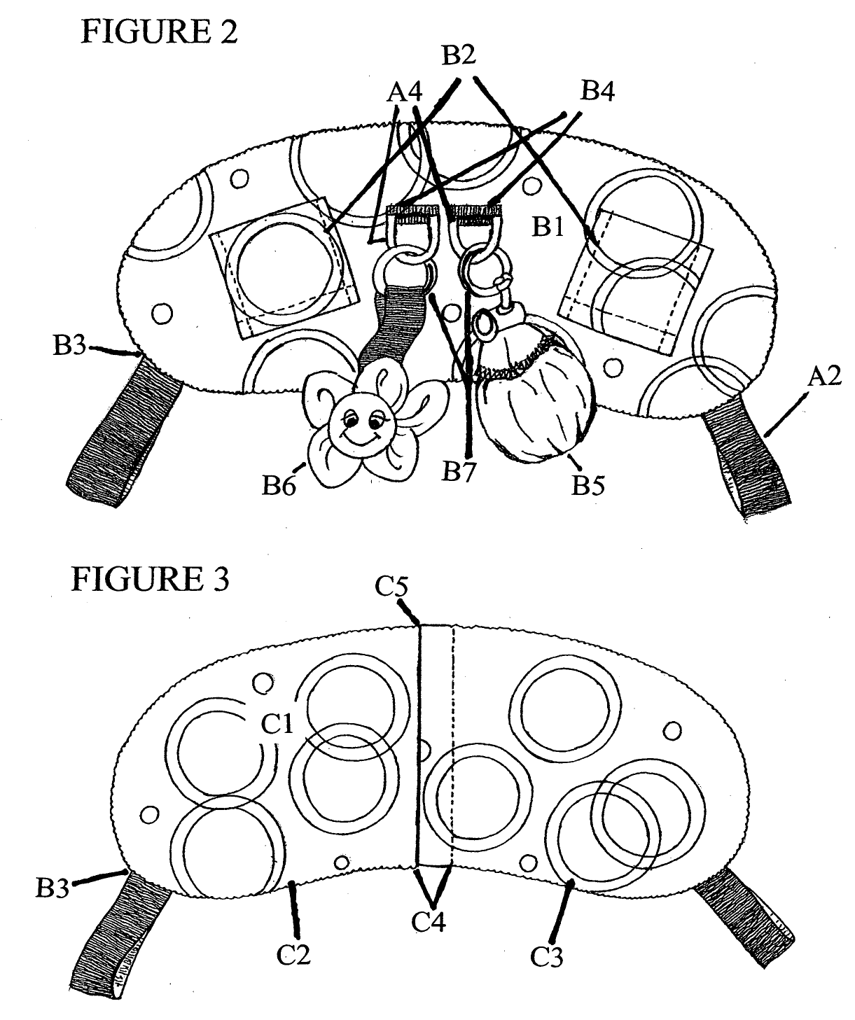

FIG. 2 illustrates the embodiment of the invention with its cover on B1, and with sensory tools attached B5/B6. The cover is form fitting and made of a pleasant washable fabric (such as fleece, cotton, or nylon) with or without a pattern. The anterior side of the cover is made from one solid panel of material B1. The posterior side is made of two overlapping panels. The panels are overlayed and sewn around the conventionally sewn or otherwise attached around the periphery. In this embodiment, the cover has two pockets B2 made of the same material, for the user to slip his hands into. These pockets are preferably pre-sewn prior to assembly. They may be symmetrical from the center, and to the outside of the anchors A4. Also visible in this figure are the slits in the seems where the handle/s come through the cover B3. This is done by pulling the handles A2 through a 1.00 inch opening in the seeming on each end of the cover B3. Item B4 identifies reinforced openings on the anterior side, through which the anchors become accessible. Central to FIG. 2, on the right side, is a typical sensory tool B5, (a drawstring pouch with a conventional stress ball), may be attached to the anchor by utilizing a 32 mm split ring key loop B7 as an attachment device. On the left side, another sensory tool (a plush colorful flower sewn to a webbing strap B6), is attached to the anchor, also by means of a 32 mm split ring key loop B7. These are but examples of sensory tools known to the art. Any other sensory tools may be employed as may be desired. The criticality lies in the ability to attach a sensory tool, however there is no criticality to the configuration of the sensory tool. The device simply needs to have a mode of attachment. Any toy of suitable size with an attachment means there-on, may be employed as a sensory tool for kids. Suitable attachable items for adults may be employed in the same manner.

FIG. 3 illustrates the posterior side of the cover C1, with the inventive device therein. The posterior side of the cover is made of two separate panels C2/C3. Visible in this figure is the vertical opening C5 of the overlapped material through which the lap pad is removed. Note, C4, there is a 1.00 inch overlap of panels C2 and C3. Also visible is B3 from the anterior view. The dotted line represents the edge of the overlapped material. As noted earlier, the opening C5 may be in the center or on any other location of the posterior cover.

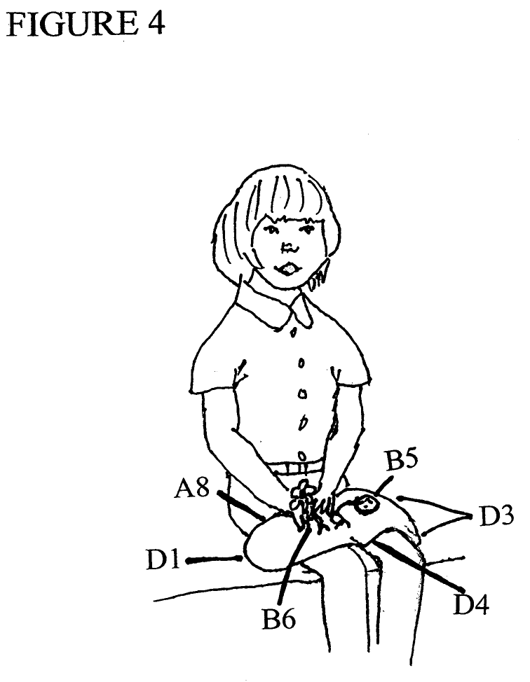

FIG. 4 illustrates the invention as it is intended to be utilized. The weighted lap pad with sensory tools attached D1 is placed in the lap of a child or other user. The narrower side of the device A8 sits snuggly against the wearer's torso. The weighted lap pad by design, hangs over the exterior side of each leg approximately 3.00 inches D3. The unit sized to the user should have between 2.00 and 5.00 inches of the device hanging over the exterior side of each leg, as per FIG. 4. It is seen in the figure that the lap pad conforms to the user D4 due to the non-visible channeling construction (as seen in FIG. 1). In the figure, the child can be seen manipulating the sensory tools B5 and B6.

It is seen that I have provided a novel kidney shaped weighted lap pad that drapes over the exterior of the legs of the user, and which has points of attachment for sensory tools of any configuration. The device may have one or more handles, which are projectable through openings in a removable cover used to prevent soiling of the device.

I claim:

1. A weighted lap pad having a body conforming configuration and a plurality of enclosed channels with a weight giving substance disposed within each of the channels; with at least one anchor means for the attachment of a sensory tool.

2. The device of claim 1 wherein channels are in a sunburst pattern

3. The device of claim 1 wherein at least one flexible handle is attached on the edge of the device.

4. A kidney shaped weighted lap pad having an anterior and posterior surfaces wherein at least one anchor means is disposed on one of said surfaces.

5. Wherein the body conforming configuration of the lap pad of claim 1 is a kidney shape.

6. The device of claim 4 having at least one flexible handle attached thereto.

7. The device of claim 1 further including a cover, having an aligned slot for each anchor present and having an opening on the opposite side of said slots for the insertion of said device into said cover.

8. The device of claim 7 further including openings aligned with the placement of the at least one handle on said device for the disposition of each handle present through the cover.

9. The device of claim 4 wherein the device has the weighted material disposed in channels and said channels are provided in a pattern divergent towards the center of the edge placed closed to the abdomen of the user.

10. A sensory tool that provides a mental stimulus to persons having sensory processing difficulties and having attachment means thereon connectible to an anchor on a weighted lap pad.

11. The sensory tool of claim 10 comprising a fabric bag with a rubber like material stress ball therein.

12. A sensory tool configured as a fabric flower having a stem with an attachment means thereon, attachable to an anchor of a weighted lap pad.

13. A weighted lap pad having channeled construction with particulated weight material disposed in said channels. With said device having at least one anchor for a sensory tool attached thereto;

14. A removable cover of a configuration matching that of the weighted lap pad, and having an elongated slot therein for the disposition of said weighted lap pad, the slot in the cover being in the location of the at least one anchor; said cover also having an aligned slot on the side opposite the elongated slot for the disposition of each of said anchors there through, and a sensory tool removable attachable to said at least one anchor of said device.

15. The device of claim 13 wherein the weighted lap pad has at least one handle and the cover has an aligned slot on the edge thereof for the disposition of said at least one handle.
